(19) United States
(12) Patent Application Publication
(10) Pub. No.: US 2008/0035159 A1
(43) Pub. Date: Feb. 14, 2008

(54) Protective lead shield for spinal surgery

(75) Inventor: Miguelangelo J. Perez-Cruet, Bloomfield, MI (US)

Correspondence Address: MILLER IP GROUP, PLC;MI4 SPINE, LLC, 42690 WOODWARD AVE., SUITE 200, BLOOMFIELD HILLS, MI, 48304, US

(73) Assignee: MI4SPINE, LLC, Bloomfield Hills, MI (US)

(21) Appl. No.: 11/500,542

(22) Filed: Aug. 8, 2006

(51) Int. Cl. A61B 19/00; A61F 5/37

(52) U.S. Cl. 128/849

(57) ABSTRACT

A protective lead shield for use during spinal surgery. The lead shield is intended to be draped over the back of a patient lying in the prone position during the surgical procedure. The lead shield includes a central opening that is positioned over the pathology being treated so as to allow the surgeon to gain access thereto with various surgical devices. The lead shield has an hour-glass shape where a narrow part of the shield is aligned with the opening. The wider ends of the shield allow the shield to be maintained in place on the patient, and the narrow part of the shield and the opening allow fluoroscopic X-rays to be taken of the spinal area being treated from both the top and the side. Thus, the surgical procedure can be effectively performed, the surgical area can be effectively X-rayed and the surgeon can be effectively protected from X-rays while the shield is in place.

### BACKGROUND OF THE INVENTION

1. Field of the Invention

This invention relates generally to a protective lead shield for surgical procedures and, more particularly, to a protective lead shield for protecting surgical personnel during surgical procedures, where the shield is draped over a patient lying in the prone position and where the shield has an opening through which the surgical procedure is performed and through which X-rays can be taken of the patient's spine.

2. Discussion of the Related Art

Many types of surgical procedures are performed on the human vertebra, such as vertebra fusion, stenosis, scoliosis correction, etc., to alleviate pain and discomfort. In many of these procedures, the patient is required to lie in a prone position face down on a surgical operating table. During the surgical procedures, the surgeon may use various instruments, such as retractors, dilators, K-wires, etc., that are inserted into the patient's spinal area around the pathology being treated. Further, various devices, such as screws, fusion bars, bone graph cages, etc., need to be mounted to the vertebra to accomplish the surgical procedure. The human vertebra includes many sensitive anatomical structures, such as the spinal cord and other neuro-components. Therefore, it is necessary that the various instruments and devices be properly positioned in the patient. A typical spinal operation will require that multiple fluoroscopic X-ray images be taken of the surgical area so that the surgeon can clearly see the progress being made during the surgery and the position of the instruments.

As is well understood, X-rays have a damaging effect on various human organs if the exposure is great enough and/or for a long enough period of time. Therefore, the surgeon and the various surgical assistants typically wear protective lead shields, such as cloaks, throat protectors, etc., during the surgical procedure. These lead shields are typically heavy, hot and cumbersome, and may decrease the surgeon's ability to safely perform the surgical procedure.

### SUMMARY OF THE INVENTION

In accordance with the teachings of the present invention, a protective lead shield is disclosed for use during spinal surgery. The lead shield is intended to be draped over the back of a patient lying in the prone position during the surgical procedure. The lead shield includes a central opening that is positioned over the pathology being treated so as to allow the surgeon to gain access thereto using the various surgical devices. The lead shield also has an hour-glass shape where a narrow part of the shield is aligned with the opening. The wider ends of the shield allow the shield to be maintained in place on the patient, and the narrow part of the shield and the opening allow fluoroscopic X-rays to be taken of the spinal area being treated from both the top and the side. Thus, the surgical procedure can be effectively performed, the surgical area can be effectively X-rayed and the surgeon can be effectively protected from X-rays while the shield is in place.

Additional features of the present invention will become apparent from the following description and appended claims, taken in conjunction with the accompanying drawings.

### BRIEF DESCRIPTION OF THE DRAWINGS

FIG. 1 is a top perspective view of a patient lying in the prone position on a surgical operating table where a lead shield of the invention is draped over the patient;

FIG. 2 is a side view of the patient lying on the surgical operating table;

FIG. 3 is a top view of the lead shield of the invention removed from the patient; and

FIG. 4 is a cut-away portion of the lead shield of the invention showing an opening in the shield that can be increased and decreased in size.

### DETAILED DESCRIPTION OF THE EMBODIMENTS

The following discussion of the embodiments of the invention directed to a protective lead shield that is used during a surgical procedure is merely exemplary in nature, and is in no way intended to limit the invention or its applications or uses. For example, the protective lead shield of the invention has particular application for use during spinal surgery. However, as will be appreciated by those skilled in the art, the lead shield of the invention will have application for other surgeries.

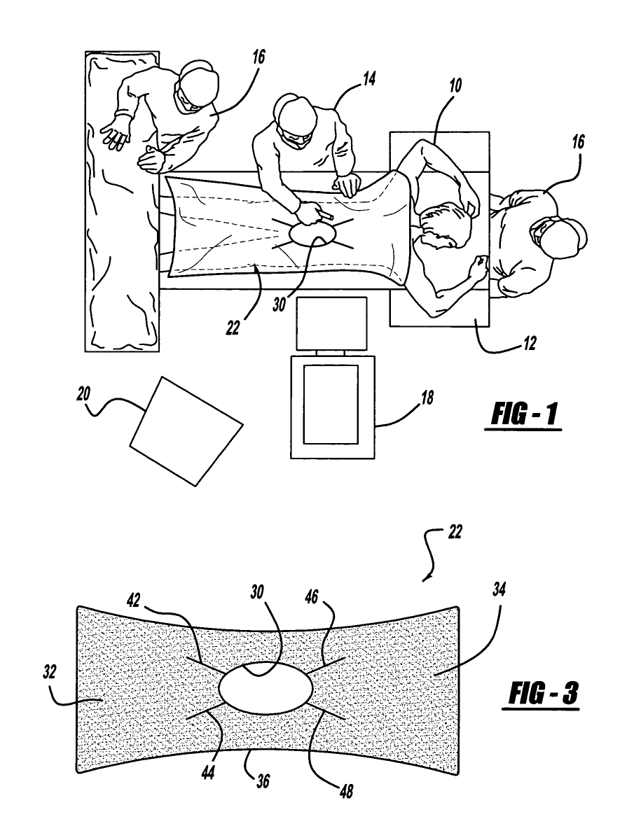

FIG. 1 is a top perspective view and FIG. 2 is a side view of a patient 10 lying in the prone position on a surgical operating table 12. A physician 14 and various assistants 16 are performing a spinal surgical procedure on the patient 10. A fluoroscopic X-ray machine 18, including a monitor 20, is used to take images of the patient's spine and other anatomy during the surgical procedure so that the surgeon 14 can ensure that various instruments, screws, retractors and other devices are accurately positioned to minimize injury to the patient 10. According to the invention, a protective lead shield 22 is draped over the back of the patient 10 to minimize exposure to the surgeon 14 and the assistants 16 from X-rays that may be scattered by anatomical structures in the body of the patient 10 and/or the various instruments in the operating room. The lead shield 22 can include any suitable number of layers including any suitable configuration of materials and have any suitable thickness for the purposes described herein.

FIG. 3 is a top view of the lead shield 22 removed from the patient 10. The lead shield 22 includes a central opening 30 having a desirable shape, here an elliptical shape. Other shapes may also be applicable. Further, the lead shield 22 has a general hour-glass shape where end portions 32 and 34 of the shield 22 are wider than a middle portion 36 of the shield 22, where the middle portion 36 aligns with the opening 30. The opening 30 is positioned relative to the patient 10 so that the surgeon 14 is able to perform the surgical procedure in relative comfort through the opening 30.

Generally, the surgeon 14 will need to see both top view X-ray images and lateral view X-ray images of the spine of the patient 10 to ensure proper progress of the surgical procedure. According to the invention, the opening 30 is large enough so that X-ray images of the surgical area of the patient 10 taken from the top are adequate. Likewise, the middle portion 36 is narrow enough so that X-ray images of the surgical area taken from the side of the patient's spine are adequate. Further, the end portions 32 and 34 are wide enough so that they drape over the sides of the patient 10 so that the shield 22 is better held in place.

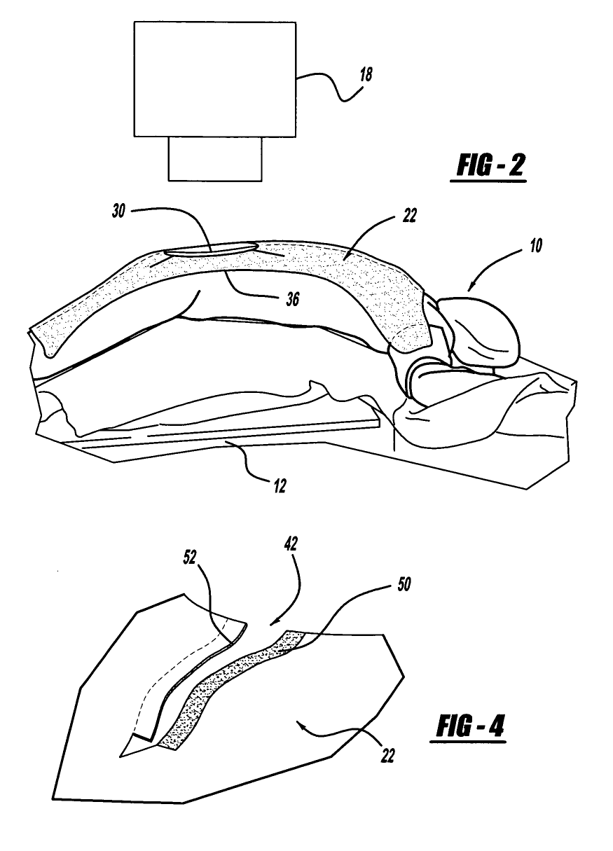

Different surgical procedures require access to a different number of vertebras. However, it is desirable to minimize the size of the opening 30 so that more X-rays are absorbed by the lead shield 22. Therefore, it is desirable that the size of the opening 30 only be large enough to expose those vertebras being operated on. According to the invention, the shield 22 includes four slits 42, 44, 46 and 48 that can be attached and detached from their respective edges to increase or decrease the size of the opening 30. In other designs, less or more slits can be employed. Any suitable technique can be used to secure together the edges of the slits 42, 44, 46 and 48 together. In one embodiment, loop and hook strips are used to close and open the slits 42-46. FIG. 4 is a cut-away enlarged view of the slit 42 that includes a loop or hook strip 50 on one side and an opposite loop or hook strip 52 on an opposite side. By selectively connecting and disconnecting the strips 50 and 52 along its length, the slit 42 can be opened and closed to increase or decrease the size of the opening 30 in combination with the other slits 44-48.

The foregoing discussion discloses and describes merely exemplary embodiments of the present invention. One skilled in the art will readily recognize from such discussion and from the accompanying drawings and claims that various changes, modifications and variations can be made therein without departing from the spirit and scope of the invention as defined in the following claims.

What is claimed is:

1. A protective shield comprising:
a body portion including an hour-glass shape where a first end portion and a second end portion of the body portion are wider than a middle portion of the body portion; and
an opening extending through the body portion at the middle portion of the body portion.

2. The shield according to claim 1 wherein the opening has an elliptical shape.

3. The shield according to claim 1 further comprising at least one device for selectively making the opening larger or smaller.

4. The shield according to claim 3 wherein the at least one device includes hook and look strips secured to opposing sides of a slit that can be selectively attached and detached.

5. The shield according to claim 3 wherein the at least one device is four devices symmetrically disposed around the opening.

6. The shield according to claim 1 wherein the body portion has a size that is effective to be positioned over the back of a person where the first and second end portions of the body portion drape over opposing sides of the person.

7. The shield according to claim 6 wherein the person is a spinal surgery patient lying in the prone position and wherein the opening is positioned relative to a spinal area being treated so as to allow a surgeon to gain access to the spinal area through the opening.

8. The shield according to claim 7 wherein the opening allows X-ray images of the spinal area to be taken through the opening from a top of the patient and the middle portion allows X-ray images to be taken of the spinal area from a side of the patient.

9. The shield according to claim 1 wherein the body portion includes lead for absorbing X-rays.

10. A protective shield comprising a body portion including a first end portion, a second end portion and a middle portion, said body portion further including an opening extending through the body portion at the middle portion.

11. The shield according to claim 10 wherein the body portion has an hour-glass shape.

12. The shield according to claim 10 wherein the opening has an elliptical shape.

13. The shield according to claim 10 further comprising at least one device for selectively making the opening larger or smaller.

14. The shield according to claim 13 wherein the at least one device includes hook and look strips secured to opposing sides of a slit that can be selectively attached and detached.

15. The shield according to claim 13 wherein the at least one device is four devices symmetrically disposed around the opening.

16. The shield according to claim 10 wherein the body portion has a size that is effective to be positioned over the back of a person where the first and second end portions of the body portion drape over opposing sides of the person.

17. The shield according to claim 16 wherein the person is a spinal surgery patient lying in the prone position and wherein the opening is positioned relative to a spinal area being treated so as to allow a surgeon to gain access to the spinal area through the opening.

18. The shield according to claim 17 wherein the opening allows X-ray images of the spinal area to be taken through the opening from a top of the patient and the middle portion allows X-ray images to be taken of the spinal area from a side of the patient.

19. The shield according to claim 10 wherein the body portion includes lead for absorbing X-rays.

20. A protective shield for use during spinal surgery, said shield including lead for absorbing X-rays, said shield comprising:
a body portion including an hour-glass shape where a first end portion and a second end portion of the body portion are wider than a middle portion of the body portion; and
an elliptical opening extending through the body portion at the middle portion of the body portion, said body portion including a plurality of slits having devices that can selectively open and close the slits to make the opening larger or smaller, wherein the body portion has a size that is effective to be positioned over the back of a patient undergoing spinal surgery where the first and second end portions of the body portion drape over opposing sides of the person, and wherein the opening allows X-ray images of a spinal area being treated to be taken through the opening from a top of the patient and the middle portion allows X-ray images to be taken of the spinal area from a side of the patient.
